(19) United States
(12) Patent Application Publication
(10) Pub. No.: US 2014/0010839 A1
(43) Pub. Date: Jan. 9, 2014

(54) METHOD OF SCREENING ANTIRETROVIRAL COMPOUNDS AND VACCINE

(71) Applicants: Monsef Benkirane, Saint Gely du Fesc (FR); Nadine Laguette, Saint Georges d'Orgues (FR); Bijan Sobhian, Saint Georges d'Orgues (FR)

(72) Inventors: Monsef Benkirane, Saint Gely du Fesc (FR); Nadine Laguette, Saint Georges d'Orgues (FR); Bijan Sobhian, Saint Georges d'Orgues (FR)

(73) Assignee: CENTRE NATIONAL DE LA RECHERCHE SCIENTIFIQUE (C.N.R.S.), Paris (FR)

(21) Appl. No.: 14/006,321

(22) PCT Filed: Mar. 23, 2012
(86) PCT No.: PCT/EP12/55237

§ 371 Date: Sep. 20, 2013

(30) Foreign Application Priority Data
Mar. 23, 2011 (FR) 11 52394

(51) Int. Cl. A61K 39/21; A61K 35/12

(52) U.S. Cl. CPC A61K 39/21; A61K 35/12
USPC 424/208.1; 435/5; 424/207.1; 435/455; 435/320.1; 424/93.21; 435/325

(57) ABSTRACT

The present invention relates to an in vitro method of screening for candidate compounds capable of being used for the preventative and/or curative treatment of a disease caused by a retrovirus in which the candidate compound which modulates the expression and/or the activity of the SAMHD1 protein is identified as a candidate compound capable of being used for the preventative and/or curative treatment of a disease caused by a retrovirus.

The invention also relates to a vaccine composition and an immunogenic composition, comprising i) an inhibitor of the expression and/or of the activity of the SAMHD1 protein and ii) at least one antigen of a retrovirus.

The human immunodeficiency virus (HIV) is the causative agent of acquired immunodeficiency syndrome (AIDS), an infectious disease that represents one of the major public health problems with which modern medicine is grappling.

According to figures published by the World Health Organisation (WHO) in 2008, the number of people living with HIV is estimated to be 33.4 million. The number of new infections by the virus is currently estimated at 2.7 million and the number of deaths caused by AIDS is about 2 million. Sub-Saharan Africa is the most affected region accounting for 67% of all cases worldwide, 68% of new cases reported amongst adults and 91% amongst children.

The main target cells for HIV are the cells of the immune system, in particular the CD4+ T lymphocytes, but also the dendritic cells, monocytes and macrophages. To the extent that the HIV interferes with the immune functions of the target cells, and its replication causes the destruction of these cells, the infection over the long term leads to the weakening of the immune system making the body vulnerable to opportunistic diseases. Thus, in the absence of antiretroviral treatment, virtually all subjects who are HIV infected progress towards AIDS, the ultimate clinical expression of the progressive destruction of the immune system.

Starting in 1996, the introduction of multi drug combination therapies implementing protease inhibitors and reverse transcriptase inhibitors has resulted in a massive reduction in morbidity and incidence of opportunistic infections, thus delaying the progression of HIV infection to the AIDS stage. However, to date, there is no treatment that would make it possible to eradicate the virus from the body and provide a permanent cure from the HIV infection.

Moreover, despite intensive research being carried out for over twenty years in the field of vaccinology, an effective curative or preventative vaccination strategy has not yet been identified.

The human immunodeficiency virus belongs to the family Retroviridae and to the genus Lentivirus. With regard to the primate lentiviruses, comparative phylogenetic analysis of human immunodeficiency viruses (HIVs) and simian immunodeficiency viruses (SIVs) has revealed the genetic similarity between HIV-1, chimpanzee SIV (SlVcpz) and the gorilla SIV (SIVgor) on the one hand, and between HIV-2 and the sooty mangabey SIV (SIVsm) on the other hand, which strengthens the hypothesis pertaining to a simian origin of HIV-1 and HIV-2 viruses.

The viruses of the genus Lentivirus differ from other genera of retrovirus by their ability to replicate both within dividing cells as well as within cells that do not divide, with this characteristic feature making these viruses good candidates for the development of lentiviral vectors that may be used in the context of gene therapies.

The genome of HIVs and SIVs consists of two homologous single stranded RNA strands, with positive polarity comprising nine genes. The three main retroviral genes common to all retroviruses are the gag, pol and env genes, which encode polyproteins that upon maturing after processing by the viral protease produce structural proteins (matrix protein, capsid protein, nucleocapsid protein, gp120 and gp41 envelope proteins) and viral enzymes involved in the replication of the virus, in its integration into the genome of the host cell and in the maturation of precursor polyproteins (respectively reverse transcriptase, integrase and protease). Two other retroviral genes encode regulatory proteins tat and rev: the tat protein is involved in the regulation of the transcription of viral genes following the integration of retroviral DNA into the cellular genome and cellular genes, while the rev protein participates in nuclear export of transcripts.

Four other genes named nef, vif, vpr and vpu encode the so called accessory proteins acting on different stages of the viral life cycle and on the regulation of different cellular pathways. In the case of HIV-2 and certain SIVs, in particular SIVsm, the gene vpu encoding the Vpu protein is absent and is replaced by the gene vpx encoding an accessory protein called Vpx.

The mature HIV/SIV virions have a spherical morphology measuring 80 nm to 120 nm in diametre and consist of a viral envelope (a phospholipid membrane in which are embedded the so called spikes consisting of three gp41 transmembrane glycoproteins covalently bonded to a trimer of the gp120 glycoprotein (Lu et al., Nature Structural Biology, 2(12): 1075-1082, 1995)) which surrounds the matrix and a cone shaped nucleocapsid. The nucleocapsid contains the viral genome, protease, reverse transcriptase, integrase, as well as some viral Vif, Vpr, Vpu and Nef (or Vpx in the case of HIV-2 and certain SIVs, in particular SIVsm) proteins.

The cycle of the HIV virus comprises of the following steps:
binding of the virus to the plasma membrane of the target cell by means of a specific interaction between the gp120 viral glycoprotein and the CD4 molecule on the cell surface;
gp120 conformational changes and binding to a coreceptor present close to CD4, namely the chemokine receptor CCR5 or CXCR4;
fusion between the viral envelope and the cytoplasmic membrane by means of gp41 involvement;
penetration of the virus into the cell and uncoating of the virus particle;
reverse transcription of the viral genomic RNA into double stranded DNA via the reverse transcriptase present in the viral particle (structure compatible with that of the cellular DNA in which the viral DNA will be integrated in order to ensure the replication of the virus and the expression thereof);
importing of the newly formed double stranded viral DNA into the cell nucleus, and then integration into the cell genome via the viral integrase (the viral DNA is then referred to as proviral DNA);
expression of the proviral DNA, formation and release of new viral particles.

Each step of the viral cycle is a potential target for the development of antiretroviral compounds. Thus it is that the first anti-HIV antiretroviral drugs developed were retroviral protease inhibitors and reverse transcriptase inhibitors. More recently, molecules inhibiting the fusion between the viral envelope and the cell membrane have been developed.

In the context of research relating to molecules capable of blocking infection by the HIV virus, the last few years have been marked by the discovery of cellular factors in primates, referred to as restriction factors, that interfere with the replication cycle of certain retroviruses including among these HIV/SIV. These restriction factors are capable of limiting the replication of HIVs/SIVs after their entry into the host cell.

The first restriction factor with demonstrated activity against HIV, namely the protein TRIM5 alpha, had been disclosed in 2004 (Stremlau et al., Nature, 427: 848-853, 2004). Stremlau and his colleagues seeking to identify the genes responsible for the resistance of some non-human primates to HIV-1, in particular the rhesus macaque, had screened a cDNA library of rhesus monkey cells, and had discovered that the simian TRIM5 alpha protein, once expressed by human cells, protected these cells against HIV-1 but not against the simian counterpart. The simian TRIM5 alpha protein does not prevent HIV-1 from penetrating into the cells, but it very quickly blocks the viral cycle.

Subsequent studies have shown that the specificity of restriction of HIV-1 by the TRIM5 alpha proteins of simian origin is carried by the proline at position 332 of the domain known as SPRY located in the C-terminal portion of the protein. The single mutation, R332P, in the SPRY domain of its human counterpart confers to the latter the ability to inhibit HIV-1.

Recently, the existence of another restriction factor for HIV has been discovered and highlighted. Thus, it has been shown that dendritic cells and cells of the monocytic lineage, in particular the macrophages, express a restriction factor that is capable of limiting their infection by certain lentiviruses, in particular HIV-1 and some SIVs. It has also been shown that the Vpx auxiliary protein expressed by HIV-2 and by some SIVs, in particular SIVsm, is capable of counteracting the action of the restriction factor, which thus explains that this restriction factor is ineffective against HIV-2 and that HIV-2 infects dendritic cells in an efficient manner right from the start of the infection.

A number of studies have been conducted in order to further characterise the mechanism of action of this restriction factor and to identify the cellular factor that is at the origin thereof (for review see Ayinde et al., Retrovirology, 7 :35, 2010).

Although the restriction factor that is counteracted by Vpx has not been identified, it has been demonstrated that this restriction factor is expressed in a constitutive manner in the cells. This restriction factor does not prevent the penetration of the virus into the cell but seems to hinder the accumulation of DNA derived from reverse transcription of the genomic RNA, the viral cycle stage also targeted by the restriction factor TRIM5 alpha. Moreover, like TRIM5 alpha, the factor inhibited by Vpx seems inducible by type 1 interferons (Gramberg et al., Curr HIV/AIDS Rep., 6: 36-42, 2009). However, this new restriction factor differs from TRIM5 alpha in several respects. While the expression of TRIM5 alpha is ubiquitous, the restriction factor inhibited by Vpx is expressed only in dendritic cells and cells of the monocytic lineage. In addition, unlike TRIM5 alpha, this restriction factor seems not to be saturable since a large excess of viral particles does not provide for restoring the infectivity of a macaque SIV that does not express a functional Vpx protein (Goujon et al., Retrovirology, 4: 2, 2007).

The restriction factor inhibited by Vpx being specifically expressed by dendritic cells and cells of the monocytic lineage, recent research studies have been focused on the implication of this restriction factor in the innate immune response mediated by these cells. During an infection, the dendritic cells play an essential role in the innate immune response, in particular by the expression and secretion of type 1 interferon. These cells are also specialised in the capture, processing and presentation of antigens, and play a central role in the immune response because they are the only cells capable of activating the naive T lymphocytes and initiating a coordinated and sustainable primary immune response from the immune system, thereby enabling the protection of the body against the infectious agent. However, in the case of HIV infection through sexual contact, in particular HIV-1, the innate immune response is weak during the acute phase (that is to say the first week following the infection), Type 1 interferons (which have antiviral activity) are generally not expressed.

Recent studies have shown that when the resistance to infection of dendritic cells is bypassed (co-infection by HIV-1 virus and virus like particles bearing Vpx), the infection by HIV-1 induces the maturation of the infected cells, an antiviral response mediated by type 1 interferons, as well as activation of T cells (Manel et al, Nature, 467(9): 214-217, 2010). It has therefore been suggested that the restriction factor inhibited by Vpx could benefit the HIV-1 escape strategy to the extent that efficient replication in dendritic cells would induce a strong innate immune response, in particular by the expression of type 1 interferon, which could allow for the establishment, right from the early post-infection days, of an effective immune response.

These studies suggest that the inefficacy of current HIV-1 vaccines would be mainly due to the restriction of infection by HIV-1 in dendritic cells and macrophages. The identification of the restriction factor expressed by these cells is therefore crucial for the development of an effective vaccine against retroviruses, lentiviruses in general, preferably against lentiviruses that do not express Vpx, and HIV-1 in particular.

Moreover, the identification of this restriction factor is important for the development of methods for gene transfer using retroviral vectors, lentiviral vectors in particular. In effect, inhibition of this restriction factor would make it possible to enhance the transduction of cells expressing this factor, in particular dendritic cells and cells of the monocytic lineage, by lentiviral vectors for the purposes of gene therapy or for in vitro gene transfer experiments.

However, in spite of numerous studies being focused on this restriction factor, its identity has remained unknown until the present time.

The inventors have now identified this restriction factor as being the protein SAMHD1.

The gene SAMHD1 (Gene ID 25939), located on chromosome 20, is transcribed into mRNA (NCBI accession number NM_{—}015474), which is translated into a SAMHD1 protein consisting of 626 residues (NCBI accession number NP_{—}056289; SEQ ID NO: 1).

The protein SAMHD1 has two identifiable regions. The first (residues 44 to 107) named “SAM” (for “Sterile Alpha Motif”), and a second (residues 162 to 335), named “HDc”, which demonstrates phosphohydrolase activity (RNase).

The protein SAMHD1 also called DCPI (for “dendritic cell-derived IFN gamma induced protein”) was identified following the screening of a cDNA library derived from dendritic cells and was revealed to be an orthologous protein in the murine (mouse) protein MG11, interferon gamma inducible protein (Li et al, Immunology Letters, 74: 221-224, 2000). SAMHD1 could be involved in the innate antiviral response (Hartman et al, J. Virol, 81: 1796-1812, 2007), and play a role in the proinflammatory response induced by TNF alpha (Lio et al., Proteomics, 8: 2640-2650, 2008). Recently, it has been demonstrated that mutations in the gene encoding SAMHD1 were associated with Aicardi-Goutières syndrome, a genetic disorder whose symptoms are similar to those encountered in a congenital viral infection (Rice et al., Nature Genetics, 41(7): 829-832, 2009; Dale et al., Am. J. Med. Genet. A., 152A(4): 938-942, 2010; Thiele et al., Hum. Mutat., 31(11): 1836-1850, 2010). It was also proposed that SAMHD1 plays a protective role in order to prevent self-activation of the innate immune response (Rice et al., Nature Genetics, 41(7): 829-832, 2009). However, the link between resistance to infection by HIV-1 of the dendritic cells and cells of the monocytic lineage and expression of SAMHD1 had never been mentioned.

The inventors have moreover also researched the region or regions involved in the restriction. They have thus demonstrated that phosphohydrolase activity of this protein is required for the restriction of HIV-1 infection. Dendritic cells expressing a mutated SAMHD1 protein within the “HDc” domain are no more resistant to the infection, the HIV-1 virus replicating strongly in these cells.

### In Vitro Method of Screening

The inventors having identified the SAMHD1 protein as the restriction factor that inhibits the replication of HIV-1, an object of the invention is to provide an in vitro method of screening for candidate compounds capable of being used for the preventive and/or curative treatment of a disease caused by a retrovirus.

This method comprises of the following steps:
contacting a candidate compound with a cell expressing SAMHD1 or with the SAMHD1 protein; and
determining whether the candidate compound modulates the expression and/or the activity of the SAMHD1 protein;
   wherein said candidate compound which modulates the expression and/or the activity of the SAMHD1 protein is identified as a candidate compound capable of being used for the preventive and/or curative treatment of a disease caused by a retrovirus.

The retrovirus may be a retrovirus belonging to a genus selected from the group consisting of the Alpharetroviruses, the Betaretroviruses, the Gammaretroviruses, the Deltaretroviruses, the Epsilonretroviruses, the Lentiviruses and the Spumaviruses.

Preferably, the retrovirus that causes the disease is a lentivirus selected from the group consisting of:
bovine lentiviruses (bovine immunodeficiency virus (BIV));
equine lentiviruses (equine infectious anemia virus (EIAV));
feline lentiviruses (feline immunodeficiency virus (FIV) and the puma lentivirus);
caprine/ovine lentiviruses (caprine arthritis encephalitis virus (CAEV), maedi visna virus (MVV)); and
primate lentiviruses (the human immunodeficiency viruses, type 1 (HIV-1), the human immunodeficiency viruses, type 2 (HIV-2) and the simian immunodeficiency viruses (SIV)).

More preferably, the retrovirus that causes the disease is a primate lentivirus that is selected from the HIV-1, HIV-2 and SIV viruses. Advantageously, it is an HIV-1 virus.

The term “to modulate the expression of the SAMHD1 protein” is understood to mean either the increase or the inhibition of the expression of this protein. Expression of the SAMHD1 protein can be modulated by modulating the transcription of the mRNA from the gene encoding SAMHD1 and/or the translation of mRNA into SAMHD1 protein (SEQ ID NO: 1) from the mRNA. In similar fashion, the term “to modulate the activity of the SAMHD1 protein” signifies enhancing or inhibiting the activity of the SAMHD1 protein.

The expression and the activity of the SAMHD1 protein can be determined in cells naturally expressing this protein, in particular, dendritic cells and cells of the monocytic lineage (in particular monocytes and macrophages), or indeed in cells that do not naturally express SAMHD1 but in which the gene or cDNA of SAMHD1 (SEQ ID NO: 2) has been introduced by genetic engineering in a manner such that it is expressed in this cell.

Techniques for introducing a polynucleotide sequence in a cell, as well as the vectors used for the implementation of these techniques are well known to the person skilled in the art, and are described, for example, by Sambrook andal. (Molecular Cloning A Laboratory Manual, Third Ed, Cold Spring Harbor Laboratory Press, Cold Spring Harbor, N.Y., 2001 Sambrook et al. (Molecular Cloning, A Laboratory Manual, Third Ed., Cold Spring Harbor Laboratory Press, Cold Spring Harbor, N.Y., 2001). Examples to be cited include the transfection of cells with an expression vector comprising the gene or cDNA of SAMHD1, in particular techniques using the coprecipitation of the expression vector with calcium phosphate or the use of various complexes of the expression vector with lipid vesicles (liposomes). The cells may be eukaryotic cells or prokaryotic cells.

Methods for determining the expression of a protein in a cell are well known to the person skilled in the art. The expression of SAMHD1 may for example be determined by quantifying the SAMHD1 protein present in the cell lysate, in particular by means of antibodies that are specific to this protein, chromatography techniques or mass spectrometry techniques. The expression of SAMHD1 may also be determined by quantifying the mRNA of SAMHD1, notably by way of quantitative RT-PCR using primers that are hybridised specifically to this mRNA.

Moreover, since the phosphohydrolase activity is essential for the activity of the protein SAMHD1, the activity of this protein may be assessed in vitro by measuring the degradation of the SAMHD1 substrate, that is to say the retoviral RNA and/or the retroviral DNA (obtained by reverse transcription), provided in known quantities. The effect of the candidate compound on the activity of SAMHD1 will thus be evaluated by measuring the substrate remaining and/or the product of degradation, and by comparing this measurement or these measurements with those obtained in the absence of the candidate compound (see for example Zimmerman et al., JMB, 378: 215-226, 2008).

In this embodiment, the SAMHD1 protein used will preferably be in purified or partially purified form. It may be produced by conventional genetic engineering techniques such as those described by Sambrook et al. (Molecular Cloning, A Laboratory Manual, Third Ed., Cold Spring Harbor Laboratory Press, Cold Spring Harbor, N.Y., 2001), for example by cloning of the gene or the cDNA encoding the SAMHD1 protein in an expression vector, and then expression by a bacterium transformed with a plasmid, in particular E. coli, or indeed transcription and then translation in vitro of SAMHD1.

In one preferred embodiment of the in vitro method of screening according to the invention:
the determination of the ability of the candidate compound to modulate the expression of SAMHD1 is carried out by quantifying the SAMHD1 mRNA and/or the SAMHD1 protein; and
the determination of the ability of the candidate compound to modulate the activity of SAMHD1 is carried out by measuring the phosphohydrolase activity of SAMHD1.

When the candidate compound tested by means of an in vitro method of screening according to the invention has an effect on the expression and/or the activity of the SAMHD1 protein, it will be advantageous to ensure that said candidate compound indeed has an effect on the replication of the retrovirus.

In order to do this, the following steps may be applied:

i) culturing in the presence of said candidate compound to be tested, the cells infected with said retrovirus, said cells being selected from the group consisting of dendritic cells, monocytes and macrophages;

ii) measuring the replication of the retrovirus;

iii) selecting as candidate compound capable of being used for the preventive and/or curative treatment of a disease caused by a retrovirus, the candidate compound in the presence of which the replication of the retrovirus which is measured in step ii) is different from those obtained in the case where the same retrovirus is cultured under the same conditions as in step i) but in the absence of the candidate compound to be tested.

In the context of the present invention, the term “gene encoding the SAMHD1 protein” is understood to mean the human gene encoding the protein having SEQ ID NO: 1, as well as genes that are homologous and orthologous and allelic variants of the gene encoding the protein having SEQ ID NO: 1. Preferably the homologous and orthologous genes and allelic variants encode a protein possessing both the “SAM” and “HDc” regions and exhibiting at least 75%, preferably at least 85%, more preferably at least 90%, 92%, 95%, 98%, 99% or 100% sequence identity with SEQ ID NO: 1.

In a similar manner, the term “SAMHD1 protein” covers the SAMHD1 protein corresponding to SEQ ID NO: 1 and to the NCBI accession number NP_{—}056289, as well as any homologous protein possessing both the “SAM” and “HDc” regions and exhibiting at least 75%, preferably at least 85%, more preferably at least 90%, 92%, 95%, 98%, 99% or 100% sequence identity with SEQ ID NO: 1.

The sequence identity percentages to which reference is made in connection with the disclosure of the present invention are determined on the basis of a global alignment of the sequences to be compared, that is to say, based on an alignment of sequences taken in their entirety, by using the Needleman-Wunsch alignment algorithm (J. Mol. Biol. 48, 443-453, 1970). This comparison of sequences may be carried out for example by making use of the software programme Needle and by using the “Gap open” parameter equal to 10.0, the “Gap Extend” parameter equal to 0.5, and a “Blosum 62” matrix. The Needle software programme is available, for example on the website ebi.ac.uk world wide, under the designation “Align”.

In the context of the present invention, the term “activity of the SAMHD1 protein” is understood to mean the inhibitory effect of SAMHD1 on the replication of HIV-1 (or of a SIV not encoding a VPX protein), with the knowledge that the phosphohydrolase activity of SAMHD1 (“HDc” domain) is responsible for this inhibitory effect. Thus, the effect on the activity of the SAMHD1 protein can be determined by measuring the effect of the candidate compound either on the viral replication or on the phosphohydrolase activity of SAMHD1.

The “candidate compound” may be any type of compound.
In the case of a candidate compound aimed at inhibiting the expression of the gene encoding SAMHD1, it may for example be interfering RNA directed against the mRNA or the pre-mRNA of SAMHD1, antisense oligonucleotides targetting the gene encoding SAMHD1, ribozymes directed against the mRNA or the pre-mRNA of SAMHD1.
In the case of a candidate compound aimed at inhibiting the activity of the protein SAMHD1, it may in particular be chemically synthesised molecules (preferably small chemical molecules) or natural molecules (for example, extracts from plants or microorganisms), antibodies or fragment of anti-SAMHD1 antibody, or even aptamers.

When it comes to chemically synthesised molecules, in particular small chemical molecules, aimed at inhibiting the activity of the SAMHD1 protein, the latter could have been pre-selected following a screening in silico (or so-called “virtual” screening), that is to say that the three dimensional structure of the “HDc” domain of SAMHD1 would have been modelled by computer, in particular the structure of the active site of the SAMHD1 phosphohydrolase, and that the molecules would have been designed developed and tested virtually by computer in a manner so as to be capable of interfering with the phosphohydrolase activity of the “HDc” domain (for example in order to block the active site).

In a similar fashion, the anti-SAMHD1 antibodies tested will preferably be directed against the “HDc” domain of SAMHD1 in order to inhibit the phosphohydrolase activity of SAMHD1.

### Vaccine and Immunogenic Composition

The inventors have shown that the inhibition of the expression of SAMHD1 would lead to the infection of cells and to the activation of dendritic cells, the latter cells secreting in particular a fairly large amount of type 1 interferon.

The ineffectiveness of current vaccines against HIV-1 is considered to be mainly due to the restriction of the infection by HIV-1 in dendritic cells and macrophages; this restriction of the specific infection of these cells prevents the development of a sufficient immune response following the inoculation of the vaccine.

A vaccine composition comprising, in addition to the immunogenic composition capable of inducing an immune response against a given retrovirus, a compound capable of inhibiting the expression and/or the activity of SAMHD1, would induce an earlier and stronger immune response against said retrovirus as the dendritic cells and the cells of the monocytic lineage would be activated early.

Thus, another object of the present invention relates to i) a vaccine composition or ii) an immunogenic composition, said compositions comprising:
an inhibitor of the expression and/or the activity of the SAMHD1 protein; and
at least one antigen of a retrovirus against which the immune response is sought to be directed or a polynucleotide sequence encoding this antigen, preferably said at least one antigen is an antigen of a lentivirus.

The inhibitors of the expression of the SAMHD1 protein and the activity of the SAMHD1 protein according to the invention are capable of reducing the expression of the SAMHD1 protein, or the activity of the SAMHD1 protein, by at least 10%, preferably by 30%, more preferably by at least 50%, and advantageously by at least 70%, 75%, 80%, 85%, 90%, 95%, or 100%.

Preferably, the inhibitor of the expression and/or the activity of the SAMHD1 protein acts by interaction with the SAMHD1 gene, the SAMHD1 mRNA or the SAMHD1 protein and not in an indirect manner.

The inhibitor is preferably selected from the group consisting of a small chemical molecule, an interfering RNA directed against the mRNA or the pre-mRNA of SAMHD1, an antisense oligonucleotide targetting the gene encoding SAMHD1, a ribozyme directed against the mRNA or the pre-mRNA of SAMHD1, an anti-SAMHD1 antibody (preferably a humanised antibody), or an anti-SAMHD1 antibody derivative, and an aptamer.

When the inhibitor acts at the level of the protein, and not at the level of the gene or the SAMHD1 transcript, it will preferably target the “HDc” domain of SAMHD1, and advantageously the active site of the phosphohydrolase domain of SAMHD1 in order to inactivate this activity.

The inhibitor may have been identified by the screening method according to the present invention.

The term “small molecule” refers to a molecule of less than 1,000 daltons in size, in particular of organic or inorganic compounds. The computer assisted design of chemical structure and the screening in silico are useful in the design of such molecules.

Here the term “anti-SAMHD1 antibody” is understood to mean an immunoglobulin molecule directed against the SAMHD1 protein, preferably against the “HDc” domain of SAMHD1 in order to inhibit the phosphohydrolase activity of SAMHD1 and to block the phosphohydrolase activity.

The term “humanised antibody” refers to an antibody produced in a non-human animal, preferably a mouse, which maintains its binding specificity to the SAMHD1 protein, but in which, in order to reduce its immunogenicity in man, the most non human sequences possible have been replaced with the corresponding human sequences. With regard to the variable domains, the sequences replaced are generally the FR (framework) regions, that is to say, the sequences located between the hypervariable loops—CDRs. Various methods for obtaining humanised antibodies are well known in and of themselves (for journal see for example ALMAGRO & FRANSSON, Frontiers in Bioscience, 13, 1619-1633, 2008).

It should be recalled that the CDRs (“Complementarity Determining Regions”) of an antibody are those portions of the variable domains which are involved in the specific recognition of the antigen. Each heavy and light chain of an antibody has three CDRs, designated VH-CDR1, VH-CDR2, VH-CDR3 in the case of the heavy chain, and VL-CDR1, VL-CDR2, VL CDR3, in the case of the light chain.

Anti-SAMHD1 antibody derivatives may be in particular:
any fragment of an anti-SAMHD1 antibody comprising at least the CDR3, and preferably further comprising the CDR2 and/or CDR1 of the heavy and light chains of said antibody;
any recombinant protein comprising an anti-SAMHD1 antibody fragment as defined here above.

Such fragments of anti-SAMHD1 antibody that may be used in accordance with the invention include in particular Fv, dsFv, Fab, Fab′2 or scFv fragments. The Fv fragments are constituted of the variable domains of the heavy and light chains VH and VL of an antibody, associated with each other by hydrophobic interactions. The dsFv fragment is constituted of a dimer VH::VL connected by a disulfide bridge. The scFv fragments are constituted of the variable portions of the heavy and light chains of an antibody, linked to each other by means of a flexible linker (CLACKSON et al., Nature, 352: 624-628, 1991), thereby forming a single-chain protein. The Fab fragments result from the action of papain on an immunoglobulin molecule, and each one contains a light chain and the first half of a heavy chain linked by a disulfide bridge. The fragment F(ab′)2 may be obtained by treatment of an antibody with pepsin: this fragment comprises of two Fab fragments and one part of the hinge region. The Fab′ fragments may be obtained from the F(ab′)2 fragments by cleavage of the disulfide bridge in the hinge region.

These fragments that bind to the antigen may also be combined in order to obtain multivalent derivatives, such as the “diabodies” or “triabodies” resulting from the combination of two or three of these fragments binding to the antigen.

The recombinant proteins comprising an anti-SAMHD1 antibody fragment may include in particular:
proteins combining at least one anti-SAMHD1 antibody fragment with at least one fragment of another antibody; examples thereof that may be cited include, bispecific immunoglobulins, conjugates of a Fab or Fv fragment of an anti-SAMHD1 antibody with a Fv or Fab fragment of an antibody of different specificity, the “bispecific diabodies” resulting from the combination of a scFv fragment of an anti-SAMHD1 antibody with a Fv or Fab fragment of an antibody of different specificity;
proteins combining at least one anti-SAMHD1 antibody fragment with a molecule that makes it possible to prolong its plasma half-life when it is administered in vivo, in particular with a water soluble polypeptide of sufficient molecular weight in order for the molecular weight of the fusion polypeptide thus obtained to be higher than the threshold of renal filtration.

Chimeric or recombinant antibodies, scFv fragments and derivatives thereof, etc may be obtained by conventional genetic engineering techniques such as those described by Sambrook et al. (Molecular Cloning, A Laboratory Manual, Third Ed., Cold Spring Harbor Laboratory Press, Cold Spring Harbor, N.Y., 2001).

The term “interfering RNA” refers to a double stranded RNA molecule capable of inhibiting in a sequence specific manner the expression of a target gene by causing the degradation of the mRNA thereof.

In order to be used in the mammalian cell, the interfering RNAs must possess a double stranded portion of less than 30 bp (base pairs) in order to avoid a non specific interferon response induced by longer double stranded RNAs. These interfering RNAs, which are RNAs containing both the target sequence as well as the corresponding antisense sequence, include in particular the small interfering RNAs (“small interfering RNAs” or siRNAs) (ELBASHIR et al., Nature, 411(6836): 494-8, 2001; TUSCHL, Nat. Biotechnol., 20(5): 446-8, 2002), the short RNAs having the shape of a hairpin (“short hairpin RNAs” or shRNAs), which are then transformed by the cellular machinery into siRNAs (PADDISON et al., Genes Dev., 16(8): 948-58, 2002; YU et al., P.N.A.S., 99(9): 6047-52, 2002; SIOLAS et al., Nat. Biotechnol., 23(2): 227-31, 2005), as well as the pre-miRNAs and miRNAs.

The siRNAs generally have a length of 21 to 25 nucleotides and they contain a double stranded portion, generally 19 to 21 nucleotides in length, constituted of the target sequence and the corresponding antisense sequence; one or the other strand, or both of them, include/s generally at the 3′ end a single stranded extension of 2 or 3 nucleotides; most often (but not necessarily), it is a dinucleotide, with TT sequence.

The shRNAs are constituted of a single strand, having a length of 50 to 70 nucleotides, which may be folded to form a structure like a hairpin, containing a double stranded portion having a length of 19 to 29 nucleotides, constituted of the target sequence and the corresponding antisense sequence, and a loop of 5 to 10 nucleotides. They may also include, at the 3′ end, a single stranded extension of 2 or 3 nucleotides.

As previously mentioned above, the Interfering RNAs can also be used in the form of precursors (pre-miRNA) or miRNA. The miRNAs are produced from pre-miRNA precursors of about 70 nucleotides that have a hairpin shape comprising a loop. The sequence of a pre-miRNA, identified under natural conditions in a eukaryotic cell, can be modified in order to introduce therein a siRNA sequence, which, upon being released by DICER will degrade a mRNA other than the one initially targeted. This modified pre-miRNA is then used as a carrier for the siRNA of interest, which results in increasing the efficacy of the interference. Advantageously, the pre-miRNA includes, from 5′ to 3′, the target antisense sequence, followed by the sequence of the loop of the natural pre-miRNA and then the target sense sequence. Preferably, the loop is that of the miR-155 pre-miRNA originating from the RNA not encoding BIC (B cell integration cluster) found in particular in mice, chicken or humans (KWAN-HO CHUNG et al., Nucleic Acids Research, 34(7), 2006).

Conventional methods for preparing nucleic acids may be implemented to produce interfering RNAs in accordance with the invention (for review see for example AMARZGUIOUI et al., FEBS Lett, 579, 5974, 2005). It may involve chemical synthesis or indeed production by genetic engineering techniques. In the latter case use would be made of an expression vector containing a DNA sequence which can be transcribed into at least one interfering RNA in accordance with the invention, placed under the control of a suitable promoter.

The “Aptamers” are a class of molecule which represents an alternative to antibodies in term of molecular recognition. Aptamers are oligonucleotide or oligopeptide sequences that have the ability to recognise practically all categories of target molecules with a high affinity and high specificity. These ligands may be isolated from a library of random sequences by implementing the “SELEX” method (for “Systematic Evolution of Ligands by EXponential enrichment”), as described in Tuerk C. and L. Gold (Science, 1990, 249 (4968): 505-10). The random sequence library may be obtained by combinatorial chemical synthesis of DNA. In this library, each member is a linear oligomer, possibly chemically modified, having a single sequence. The possible modifications, the uses and advantages of this class of molecules have been disclosed by Jayasena S D, Clin. Chem., 1999, 45 (9):1628-50. Peptide aptamers are conformational peptides, generally consisting of around twenty residues selected from combinatorial libraries by means of the two hybrid system (Colas et al., Nature, 1996, 380: 548-50; Hoppe-Seyler et al., J. Steroid Biochem. Mol. Biol., 2001, 78(2): 105-11)).

The term “ribozyme” refers to an RNA molecule having an enzymatic activity that is capable of cleaving other distinct RNA molecules, with the cleavage being specific to a given target ribonucleotide sequence. In the present case, the target ribonucleotide sequence is included in the sequence of the mRNA or the pre-mRNA derived from the transcription of the gene encoding the SAMHD1 protein.

The vaccine composition or the immunogenic composition according to the invention will include at least one antigen of the retrovirus against which it is sought to direct the vaccine, or the polynucleotide sequence encoding this antigen.

Where the composition comprises a polynucleotide sequence encoding an antigen of the retrovirus, said sequence will be included in any expression vector allowing the expression of the antigen when the vector is in a eukaryotic cell.

The antigen of a retrovirus against which it is sought to direct the selected vaccine or the immune response will preferably be from the group consisting of a protein of said retrovirus or a fragment thereof, an expression vector comprising the polynucleotide sequence encoding a protein of said retrovirus or a fragment thereof, a virus like particle possessing the structural proteins of said retrovirus, a lipopeptide antigen comprising proteins or peptide fragments of said retrovirus, said retrovirus in its inactivated form or in its attenuated form, and a viral vector. Where the antigen is a viral protein, it will preferably be a structural protein selected from the group consisting of envelope glycoproteins (gp120 and gp41 in the case of HIVs), the capsid protein, the matrix protein, more preferably envelope glycoproteins.

The expression vectors comprising the polynucleotide sequence encoding this antigen include in particular the expression vectors described in the review article by Kumar P. et al., Curr. Gene Ther., 11 (2): 144-53, 2011. These expression vectors may in particular be: a retroviral expression vector, adenoviral vector, etc.

Preferably, the retroviral vector will be a lentiviral vector of the type HIV, SIV and FIV, given the fact that these vectors have the property of being able to transduce cells that do not divide such as monocytes and dendritic cells, the main targets of the vaccine since it is these cells that it is sought to activate early. The lentiviral vector will preferably be an HIV vector. This type of lentiviral vector is described in particular in the journal article by Kumar P. et al., Curr. Gene Ther., 11 (2): 144-53, 2011). Preferably, it will be a lentiviral vector of the type pTRIP such as that previously described by Royer-Leveau et al. (Journal of Virological Methods, 105: 133-140, 2002).

The term “virus like particle” is understood to mean viral particles (comprising at least the structural proteins of retroviruses) without genome. The techniques for obtaining virus like particles are well known to the person skilled in the art. The technique to be mentioned in particular consists of inducing expression of a vector encoding the structural proteins of the retrovirus in a cell, in particular in a cell of a mammal, insect, yeast, or bacteria.

The term “retrovirus in its inactivated form” is understood to mean any viral particle that is incapable of infecting a target cell that is normally susceptible and permissible to the infection or to carry out a replication cycle in the target cell that is normally susceptible and permissible to the infection.

Retroviruses in the inactivated form may be obtained by techniques that are well known to the person skilled in the art. Among them are retroviruses inactivated by treatment with aldrithiol-2 or inactivated by heat (for example two times×30 minute periods at 56° C.).

The term “retrovirus in its attenuated form” is understood to mean any retrovirus wherein the ability to complete a viral replication cycle is lower than that of the same native retrovirus. In order to obtain an attenuated retrovirus, it is possible to delete certain genes of the retrovirus, in particular the accessory genes: for example, in the case of the HIV virus it is possible to remove from the viral genome the genes nef, vif, vpr and/or vpu so as to attenuate the virus (see Watkins DI. Top HIV Med; 18(2): 35-6, 2010).

The term “lipopeptide antigen comprising proteins or peptide fragments of the retrovirus” is understood to mean a protein of the retrovirus or a fragment thereof which has been modified at one of the ends by the addition of a lipid group. Preferably, the lipid group is an N-ε-palmitoyl-lysylamide group. The lipopeptide antigens are for example, those described in the publication Cobb et al. (Journal of Immunological Methods, 365: 27-37, 2011).

The term “pseudotyped viral vector” is understood to mean a viral particle wherein the envelope glycoproteins are derived from the retrovirus against which it is sought to direct the vaccine while the rest of the virus, in particular the internal constituent components, are derived from another virus.

In a preferred embodiment, the vaccine composition and the immunogenic composition according to the invention would include a retrovirus in its attenuated form or a retroviral vector, as well as at least one inhibitor of the expression and/or the activity of the SAMHD1 protein.

The retrovirus against which it is sought to direct the vaccine composition or the immunogenic composition may be a retrovirus belonging to a genus selected from the group consisting of the Alpharetroviruses, the Betaretroviruses, the Gammaretroviruses, the Deltaretroviruses, the Epsilonretroviruses, the Lentiviruses and the Spumaviruses.

Preferably, it is a lentivirus selected from the group consisting of:
bovine lentiviruses (bovine immunodeficiency virus (BIV));
equine lentiviruses (equine infectious anemia virus (EIAV));
feline lentiviruses (feline immunodeficiency virus (FIV) and the puma lentivirus);
caprine/ovine lentiviruses (caprine arthritis encephalitis virus (CAEV), maedi visna virus (MVV)); and
primate lentiviruses (the human immunodeficiency viruses, type 1 (HIV-1), the human immunodeficiency viruses, type 2 (HIV-2) and the simian immunodeficiency viruses (SIV)).

The lentivirus is more preferably an HIV, advantageously an HIV-1.

The antigen will be selected such that it is of the same origin as the retrovirus against which it is sought to direct the vaccine composition or the immunogenic composition.

Preferably, the antigen will be a lentivirus antigen, advantageously of HIV-1.

The vaccine composition and the immunogenic composition may be in any form that may be suitable for administration. Typically, the immunogenic compositions and vaccines according to the invention are prepared in injectable forms (either in the form of a liquid solution, or in the form of a suspension), solid forms suitable for being suspended or dissolved in a liquid before injection.

Pharmaceutically acceptable excipients, that is to say, which have no adverse physiological effect for the subject, and are compatible with the survival of mycobacteria present in the vaccine, may be used. Included amongst these excipients to be cited are water, a saline solution (physiological water), glycerol.

The vaccine or immunogenic composition may also contain minor amounts of auxiliary substances, such as pH buffering agents, or adjuvants which serve the function of improving the efficacy of the vaccine by stimulating, activating, extending, enhancing, potentiating and/or modulating the immune response directed against the mycobacterium present in the vaccine. The adjuvants used in human medicine, in particular those commonly used in the case of a live attenuated vaccine, are well known to the person skilled in the art. In particular mention may be made of aluminum hydroxide or aluminum phosphate, squalene, tocopherol, hydroxyapatite, complete and incomplete Freund's adjuvants, dimethyldioctadecylammonium bromide (DDA), lymphokines (in particular IFN-gamma, IL-2 and IL-12), the cell wall derived from M. bovis BCG strain.

### Modulator of the Expression and/or the Activity of SAMHD1

The present invention also relates to a modulator of the expression and/or the activity of SAMHD1 for use thereof in the treatment and/or prevention of a disease by a retrovirus.

The term “modulator of the expression of the SAMHD1 protein” is understood to mean any compound capable of either increasing or inhibiting the expression of this protein. The term “expression of the SAMHD1 protein” is understood to mean the transcription of the mRNA from the gene encoding SAMHD1 or the translation of the mRNA into SAMHD1 protein (SEQ ID NO: 1) from the mRNA. Similarly, the term “modulator of the activity of the SAMHD1 protein” is understood to mean amplifying or inhibiting the activity of the SAMHD1 protein.

Modulators of the expression and/or activity of SAMHD1 may be isolated from the methods of screening according to the invention.

When the modulator of the expression and/or the activity of SAMHD1 is an inhibitor of the expression and/or the activity of SAMHD1, it is preferably an inhibitor such as defined here above in the vaccine composition, that is to say an inhibitor selected from the group consisting of a small chemical molecule, an interfering RNA directed against the SAMHD1 mRNA or pre-mRNA, an antisense oligonucleotide targetting the gene encoding SAMHD1, a ribozyme directed against the SAMHD1 mRNA or pre-mRNA, an anti-SAMHD1 antibody (preferably a humanised antibody), or an anti-SAMHD1 antibody derivative, and an aptamer.

When the inhibitor acts at the level of the protein and not at the level of the gene or the SAMHD1 transcript, it will preferably target the “HDc” domain of SAMHD1, and advantageously the active site of the phosphohydrolase domain of SAMHD1 in order to inactivate this activity.

The modulator of the expression and/or the activity of SAMHD1 is used in a therapeutically effective amount. In a subject already infected with a retrovirus, the term “therapeutically effective amount” is understood to mean an amount of compound sufficient so as to prevent the disease from progressing to an aggravated level, or sufficient so as to cause the regress of the disease. In a non infected subject, the “therapeutically effective amount” is the amount that is sufficient to protect a subject who is brought into contact with said retrovirus and to prevent the occurrence of the disease caused by this retrovirus. The exact amount of the modulator to be administered varies depending on the age and weight of the subject to be treated, the type of disease, the stage of disease, the condition of the subject, the mode of administration, the frequency of administration and as well as the other ingredients present in the composition that includes the inhibitor.

The disease is caused by a retrovirus that is selected from the group consisting of the Alpharetroviruses, the Betaretroviruses, the Gammaretroviruses, the Deltaretroviruses, the Epsilonretroviruses, the Lentiviruses and the Spumaviruses.

Preferably, the disease is caused by a retrovirus which is a lentivirus selected from the group consisting of:
bovine lentiviruses (bovine immunodeficiency virus (BIV));
equine lentiviruses (equine infectious anemia virus (EIAV));
feline lentiviruses (feline immunodeficiency virus (FIV) and the puma lentivirus);
caprine/ovine lentiviruses (caprine arthritis encephalitis virus (CAEV), maedi visna virus (MVV)); and
primate lentiviruses (the human immunodeficiency viruses, type 1 (HIV-1), the human immunodeficiency viruses, type 2 (HIV-2) and the simian immunodeficiency viruses (SIV)).

More preferably, the retrovirus that causes the disease is a primate lentivirus that is selected from the HIV-1, HIV-2 and SIV viruses. Advantageously, it is an HIV-1 virus.

### Retroviral Transduction and Transduction Kit

The inventors have shown that the SAMHD1 protein is responsible for the inhibition of the replicative cycle of HIV-1 in cells expressing this protein, in particular in the dendritic cells and the cells of the monocytic lineage. Thus, inhibition of the expression and/or the activity of the SAMHD1 protein makes it possible to increase the efficiency of the transduction of these cells by retroviral vectors.

Accordingly, the object of the present invention also relates to the in vitro or ex vivo use of an inhibitor of the expression and/or the activity of the SAMHD1 protein in order to increase the transduction of cells expressing SAMHD1 bp a retroviral expression vector, preferably a lentiviral expression vector, more preferably an HIV-1 expression vector.

Another object of the present invention relates to a kit for transducing a retroviral expression vector, preferably a lentiviral expression vector, in a cell expressing SAMHD1, preferably a cell from the monocytic cell lineage or a dendritic cell, comprisingf:
an inhibitor of the expression and/or the activity of the SAMHD1 protein;
a retroviral expression vector, preferably a retroviral expression vector that does not naturally express the Vpx protein, more preferably even a lentiviral expression vector which does not naturally express the Vpx protein, more preferably an HIV-1 expression vector.

The inhibitor present in the kit may be any inhibitor as described previously in this present patent application.

In a preferred embodiment of the in vitro or ex vivo use and of the kit according to the invention, the retroviral expression vector used encodes a protein of interest that is sought to be expressed in the cell expressing SAMHD1.

In the context of the present invention, the term “transduction” or “transduce” is understood to mean the transfer of the genomic RNA of a retrovirus into the host cell upon being infected by a retrovirus, followed by the reverse transcription of the genomic RNA into double stranded DNA capable of being integrated into the genome of the host cell, and as necessary, of expressing a protein of interest, if the vector encodes such a protein.

The retroviral expression vector may be any retroviral vector, in particular a retroviral vector selected from the group consisting of the Alpharetroviruses, the Betaretroviruses, the Gammaretroviruses, the Deltaretroviruses, the Epsilonretroviruses, the Lentiviruses and the Spumaviruses. The retroviral expression vector is preferably a lentiviral expression vector selected from the group consisting of:
bovine lentiviruses (bovine immunodeficiency virus (BIV));
equine lentiviruses (equine infectious anemia virus (EIAV));
feline lentiviruses (feline immunodeficiency virus (FIV) and the puma lentivirus);
caprine/ovine lentiviruses (caprine arthritis encephalitis virus (CAEV), maedi visna virus (MVV)); and
primate lentiviruses (the human immunodeficiency viruses, type 1 (HIV-1), the human immunodeficiency viruses, type 2 (HIV-2) and the simian immunodeficiency viruses (SIV)).

The lentiviral expression vector is most preferably a human immunodeficiency virus type 1 (HIV-1), a human immunodeficiency virus type 2 (HIV-2) or a simian immunodeficiency virus (SIV). Advantageously, it is an HIV-1.

Preferably, the SAMHD1 expressing cells are selected from the group consisting of dendritic cells and cells of the monocytic cell lineage, in particular monocytes and macrophages.

The present invention also relates to a method for expressing a protein of interest in cells expressing SAMHD1, this method comprising the following steps of:

i) placing the cells expressing SAMHD1 with an inhibitor of the expression and/or activity of the SAMHD1 protein;

ii) infecting the cells expressing SAMHD1 from step i) with a retroviral expression vector encoding a protein of interest, preferably a lentiviral expression vector;

iii) optionally, checking to ensure that the cells expressing SAMHD1 do indeed express the protein of interest, for example by Western blot or immunofluorescence testing making use of antibodies specific to the protein of interest.

In one embodiment, the steps i) and ii) are carried out simultaneously.

In a particular embodiment of the method according to the invention, cells expressing SAMHD1 were obtained from a subject, for example from the peripheral blood of the subject. They can for example be isolated by leukapheresis (see Cobb et al., Journal of Immunological Methods, 365: 27-37, 2011), or sorted by flow cytometry, in particular on the basis of their particle size and/or surface markers specific to these cells. When it comes to dendritic cells, the specific markers include in particular CD86, CD80, CD83. In the case of monocytes and macrophages, the CD14 marker should in particular be mentioned.

In an advantageous embodiment, the method comprises a final step in which the cells expressing SAMHD1 derived from a subject obtained in step ii) (or iii)) are reimplanted in said subject. This method is particularly interesting in the context of gene therapy aimed at inducing expression of a protein of interest in the subject by cells of the subject expressing SAMHD1, preferably by dendritic cells and/or cells of the monocytic lineage.

Another object of the present invention relates to the use of cells expressing SAMHD1 encoding a protein of interest obtainable by the method for expressing a protein of interest according to the invention for use thereof as a medicament for treating and/or preventing a disease in respect to which the expression of the protein of interest enables prevention thereof, for healing, decreasing the symptoms of the said disease and/or improving the health of a patient suffering from this disease.

The protein of interest may notably be an antigen of a microorganism, for example a bacterial antigen, viral or yeast derived antigen, which would make it possible to treat or prevent a disease in which the microorganism is involved. The protein could also be an antigen specifically expressed by cancer cells, the cells would then be used within the scope of an antitumor therapy.

### Induction of the Expression of Type 1 Interferons

The inventors have now shown that the inhibition of SAMHD1 expression by interfering RNA in dendritic cells, in the absence of any infection, activated the expression and secretion of type 1 interferons.

The term “type1 interferons” is understood to mean alpha and beta interferons.

Thus, an object of the present invention relates to the use in vitro or ex vivo of an inhibitor of the expression and/or the activity of the SAMHD1 protein (SEQ ID NO: 1) so as to induce the expression of type 1 interferons in cells naturally expressing the SAMHD1 protein.

Moreover, since type 1 interferons are involved in the innate antiviral immune response, the invention also relates to a compound selected from i) an inhibitor of the expression of the SAMHD1 protein, and ii) an inhibitor of the activity of the SAMHD1 protein, for use as a medicament for inducing expression of type 1 interferons in cells naturally expressing the SAMHD1 protein, preferably for inducing an innate immune response during infection viral, in particular a retrovirus infection, for example an infection by HIV virus.

The cells naturally expressing the SAMHD1 protein are preferably dendritic cells and/or cells of the monocytic cell lineage.

### Induction of Resistance to Infection by a Retrovirus

Another object of the invention relates to a method in vivo, in vitro or ex vivo for inducing resistance to infection by a retrovirus, preferably a retrovirus of the genus lentivirus, in a cell that does not naturally express the SAMHD1 protein, said method comprising the following steps of:
introducing a sequence encoding SAMHD1 protein in said cell that does not naturally express the SAMHD1 protein;
optionally verifying the expression of SAMHD1; the cells expressing the SAMHD1 protein being resistant to infection by a retrovirus.

The sequence encoding the SAMHD1 protein may be the gene or cDNA encoding SAMHD1.

In the context of the present invention, the term “resistance to infection by a retrovirus” is understood to mean a reduction in the replication of retroviruses in the cell in comparison with the rate of replication observed in a similar cell not expressing SAMHD1 (that is to say a cell in which the gene encoding SAMHD1 had not been introduced). Preferably, the decrease in the replication of the retrovirus will be at least 10%, preferably 30%, more preferably at least 50%, and advantageously at least 70%, 75%, 80%, 85%, 90%, 95%, or 100%.

The replication of the retrovirus could be measured by techniques that are well known to the person skilled in the art. For example, measuring the replication of a retrovirus can be carried out by means of quantitative PCR using primers that are hybridised specifically to the sequence of said retrovirus, by the quantification of reverse transcriptase activity and/or by the quantification of viral antigens.

The present invention also relates to a method for treating or preventing infection by a retrovirus in a subject, the method comprising the following steps of:

i) introducing a sequence encoding the SAMHD1 protein in a cell of the subject to be treated, said cell not naturally expressing the SAMHD1 protein and being the target cell of the retrovirus during infection or a cell that is capable of differentiating into a target cell of the retrovirus;

ii) optionally controlling the expression of SAMHD1 in cells in which the sequence of the gene encoding SAMHD1 has been introduced, the SAMHD1 protein expressing cells being resistant to infection by a retrovirus; and

iii) reimplanting the cell expressing SAMHD1 obtained in step i) (or ii)) in the subject.

The term “target cell of the retrovirus during infection” is understood to mean any cell that is naturally infected by the virus. For example, in the case of infection by the HIV virus, the CD4+ T lymphocytes would in particular mentioned.

The term “reimplanting” refers to the administration of the cell to the subject. In the case of CD34+ stem cells, it refers to reconstitution by CD34+ cells reinfused into the bone marrow of the patient prepared to receive these cells (see the review article by Serrano et al., Curr HIV/AIDS Rep., 7(3): 175-84, 2010).

Another object of the present invention relates to a cell not naturally expressing the SAMHD1 protein in which the sequence encoding the SAMHD1 protein has been introduced, said cell being a target cell of the retrovirus during infection or a cell that is capable of differentiating into a target cell of the retrovirus, for use thereof as a medicament for treating or preventing a disease that is caused by a retrovirus in a subject.

In a particularly advantageous embodiment, the cell is an autologous cell, that is to say, that it is derived from the subject for whom the cell will be used as a medicament for treating or preventing a disease caused by a retrovirus. Thus, the present invention also relates to a cell of a subject that does not naturally express the SAMHD1 protein in which the sequence encoding the SAMHD1 protein has been introduced, said cell being a target cell of the retrovirus during infection or a cell that is capable of differentiating into a target cell of the retrovirus, for use thereof as a medicament for treating or preventing a disease caused by a retrovirus in this subject.

The subject would preferably be a human or non human mammal, in particular a non human primate, a cat, a goat.

In general terms, the subject will be chosen based on the retrovirus and host specificity of this retrovirus. For example, for the HIV virus, the subject would be human, for the FIV the subject would be a cat, etc.

Within the context of the method in vivo, in vitro or ex vivo for inducing resistance to infection by a retrovirus, and the method for treating or preventing an infection by a retrovirus in a subject, and the uses of a cell not naturally expressing the SAMHD1 protein in which the sequence encoding the SAMHD1 protein has been introduced as above, the retrovirus will in particular be a retrovirus belonging to a genus selected from the group consisting of the Alpharetroviruses, the Betaretroviruses, the Gammaretroviruses, the Deltaretroviruses, the Epsilonretroviruses, the Lentiviruses and the Spumaviruses. The retrovirus is preferably a lentivirus selected from the group consisting of:
bovine lentiviruses (bovine immunodeficiency virus (BIV));
equine lentiviruses (equine infectious anemia virus (EIAV));
feline lentiviruses (feline immunodeficiency virus (FIV) and the puma lentivirus);
caprine/ovine lentiviruses (caprine arthritis encephalitis virus (CAEV), maedi visna virus (MVV)); and
primate lentiviruses (the human immunodeficiency viruses, type 1 (HIV-1), the human immunodeficiency viruses, type 2 (HIV-2) and the simian immunodeficiency viruses (SIV)).

The lentivirus is more preferably a human immunodeficiency virus type 1 (HIV-1), a human immunodeficiency virus type 2 (HIV-2), or a simian immunodeficiency virus (SIV). Advantageously, it is an HIV-1 or SIV virus that does not naturally express the Vpx protein.

Preferably, the cell not naturally expressing the SAMHD1 protein is a hematopoietic stem cell capable of differentiating i) into lymphoid stem cells which would in turn divide to give lymphoid cells, in particular CD4+ T lymphocyte cells, CD8+ T lymphocyte cells, B lymphocyte cells, plasmoid dendritic cells; and ii) into myeloid stem cells which would in turn divide to give cells of the myeloid cell lineage, in particular monocytes. Hematopoietic stem cells are particularly characterised by the expression of immunological markers CD34 and Thy1 (CD34+ and Thy1+ cells) and the absence of expression of the marker CD33 (CD33− cells). These cells may be isolated by techniques well known to the person skilled in the art, especially from bone marrow cells, but also from peripheral blood by sorting the cells by flow cytometry on the basis of surface markers, in particular CD34.

In a preferred embodiment, the introduction of the sequence encoding the SAMHD1 protein in said cell not naturally expressing the SAMHD1 protein is carried out by means of a gene transfer vector commonly used in gene therapy. Particularly noteworthy among these are viral vectors such as adenoviral and retroviral vectors and non-viral vectors such as plasmid expression vectors (Elsabahy et al, Curr Drug Deliv, 2011 February 3, PMID: 21291381).

Where it is desired that the gene encoding the SAMHD1 protein be integrated into the genome of the host cell, a retroviral vector is to be used, preferably a lentiviral expression vector.

Another object of the invention relates to cells naturally expressing or not naturally (or weakly) expressing the SAMHD1 protein wherein the SAMHD1 sequence encoding the protein has been introduced and which thus expresses a recombinant SAMHD1 protein. Preferably, the cells are cells that do not naturally express the SAMHD1 protein or that naturally express the protein albeit only weakly: particularly to be mentioned are the CD4+ T lymphocyte cells and stem cells.

The introduction of the sequence encoding the SAMHD1 protein in the said cells naturally expressing or not naturally (or weakly) expressing the SAMHD1 protein is performed using expression vectors well known to the person skilled in the art, for example, a retroviral vector containing this sequence.

Preferably, the cell not naturally (or weakly) expressing the SAMHD1 protein is a hematopoietic stem cell capable of differentiating i) into lymphoid stem cells which would in turn divide to give lymphoid cells, including particularly CD4+ T lymphocyte cells, CD8+ T lymphocyte cells, B lymphocyte cells, plasmoid dendritic cells; and ii) into myeloid stem cells which would in turn divide to give cells of the myeloid cell lineage, in particular monocytes.

The present invention will be better understood with the help of the additional description that follows, which refers to non-limiting examples showing that inhibition of the expression of SAMHD1 in dendritic cells induces the expression of type 1 interferons while on the other hand, also rendering these cell more susceptible to infection by HIV-1.

### DESCRIPTION OF FIGURES

FIG. 1. A shows an immunoprecipitation performed on lysates of THP-1 cells expressing (lane 2) or not expressing (lane 1) Vpx fused with Flag and HA peptides using successively anti-Flag and then anti-HA antibodies, and coupled with elutions under native conditions by competition with an excess of Flag and HA peptide. The proteins thus eluted were analysed by Western blotting and using anti-SAMHD1 antibodies. FIG. 1. B shows the Western blot analysis using anti-SAMHD1, Flag or Tubulin antibodies, of total cell extracts (“WCE” Whole cell extracts) prepared from cells expressing (lane 4) or not expressing (lane 3) eVpx.

The FIG. 2. A is a histogram showing the infection by an HIV-1 expressing luciferase (HIV-LUC-G) (infection measured by quantification of luciferase activity) of THP1 cells naturally expressing SAMHD1 that had been transduced with an SAMHD1 non specific control shRNA (Scr) or an SAMHD1 specific shRNA (sense strand 5′CGCAGGAUGGCGAUGUUAU3′; antisense strand 5′AUAACAUCGCCAUCCUGCG3′) to inhibit its expression. The FIG. 2. B is a histogram showing the infection by HIV-LUC-G of THP1 cells naturally expressing SAMHD1 that had been transduced with i) an SAMHD1 non specific control shRNA (Scr), ii) an SAMHD1 specific shRNA (Samhd1), or iii) an SAMHD1 specific shRNA (Samhd1) and vector encoding a form of SAMHD1 resistant to RNAi (Samhd1-R)

The “NI” columns represent non infected cells.

FIG. 3 is a histogram showing the involvement of the “HDc” domain of the SAMHD1 protein in the inhibition of HIV infection. The “WT” columns show the results obtained when HeLa cells (permissive to HIV-1) overexpress SAMHD1. The “HD/AA” columns show the results obtained when HeLa cells overexpress a mutated form of SAMHD1 not exhibiting phosphohydrolase activity. The “HIV-LUC-G” columns represent the results obtained during infection of cells with an HIV-1 expressing luciferase. The “NI” columns represent non infected cells.

The FIG. 4. A is a histogram showing the infection of primary dendritic cells derived from a healthy donor (“HD”) by an HIV-1 when the cells were treated or not with siRNAs specifically targetting SAMHD1 (measurement carried out by quantification of the number of cells expressing the viral protein p24). The “Scr” column represents the result obtained when the cells were treated with an SAMHD1 non specific control siRNA, while the columns “1” and “2” represent the result obtained when the cells were treated with two different siRNAs specifically targetting SAMHD1 (siRNA corresponding respectively to SEQ ID Nos: 3 and 4). The FIG. 4. B is a histogram showing the infection of primary dendritic cells derived from healthy donors “HD1” and “HD2” by a lentiviral vector pTRIP when these cells were treated or not with siRNA specifically targetting SAMHD1. The “Scr” column represents the result obtained when the cells were treated with an SAMHD1 non specific control siRNA; the “Dynamin 2” column represents the result obtained when the cells were treated with a dynamin 2 specific siRNA, while the columns “1” and “2” represent the result obtained when the cells were treated with two different siRNAs specifically targetting SAMHD1.

### EXAMPLE 1

### Materials and Methods

The THP-1 stable cell lines stably expressing Vpx tagged with HA and Flag peptides (hereinafter abbreviated as eVpx) were generated by way of using a retroviral vector MMLV (Moloney Murine Leukemia Virus) that contains a bicistronic transcription unit enabling the expression of the protein of interest and a selection marker (the a chain of the IL2-IL2Rα receptor).

The cells transduced with the retroviral vector were selected with magnetic beads coupled with an antibody that recognises IL2Rα.

Cell extracts making it possible to observe the effect of Vpx on SAMHD1 degradation were performed with a lysis buffer containing 0.5% Triton, 150 mM of NaCl, 10 mM of KCL, 1.5 mM of MgCl2, 0.5 mM of EDTA, 10 mM of β-mercaptoethanol and 0.5 mM of PMSF.

The eVpx immunoprecipitations were performed with the use of an antibody specifically recognising the Flag or HA tag. The immunoprecipitates were eluted under native conditions with an excess of HA or Flag peptide.

The infections were carried out either i) with an HIV virus containing the gene encoding Luciferase in place of the gene encoding Nef pseudotyped with the envelope glycoprotein of the vesicular stomatitis virus (HIV-LUC-G); or ii) with a wild HIV virus pseudotyped with the envelope glycoprotein of the vesicular stomatitis virus (HIV-G).

The virus doses used to perform the infections were determined by assay of the p24 protein (Gag component) contained in the virus producing cells and standardised before the infections.

For experiments with the HIV-LUC-G, doses of 1 ng (for HeLa cells) or 100 ng (for THP-1 cells) were used. For experiments with the HIV-G a dose of 100 ng was used.

The RNA interference experiments have been performed using siRNAs corresponding to SEQ ID Nos: 3 (5′GAUUCAUUGUGGCCAUAUA3′) and 4 (5′CAACCAGAGCUGCAGAUAA3′) (in primary dendritic cells) or a shRNA (sense strand 5′CGCAGGAUGGCGAUGUUAU3′ (SEQ ID NO: 5); antisense strand 5′AUAACAUCGCCAUCCUGCG3′ (SEQ ID NO: 6) (in THP-1 cells); specifically targetting SAMHD1. Stable THP-1 cell lines were established by means of a puromycin selection marker included in the vector encoding shRNA.

For experiments carried out on primary dendritic cells derived from the healthy donor (HD) the extinguishing of the expression of SAMHD1 was carried out on a transitory basis.

SAMHD1 mutants were generated in accordance with the standard protocol for site directed mutagenesis. These mutants as well as the sequence encoding wild SAMHD1 were introduced into a retroviral vector.

The expression of wild SAMHD1 as well as the mutants derived therefrom was conducted by means of transduction using these retroviral vectors.

The primary dendritic cells were purified from peripheral blood mononuclear cells of healthy donors by CD14 sorting followed by stimulation with cytokines (GM-CSF and IL-4) for 6 days prior to their experimental use.

### EXAMPLE 2

### Results

The literature shows the existence of an unidentified cellular factor expressed in dendritic cells and macrophages, that is capable of restricting infection by lentiviruses and in particular by HIV-1. The action of this factor is counteracted by the viral protein Vpx expressed by the HIV-2 and by a certain strain of SIV. The Vpx protein induces the degradation of this restriction factor by targetting it for proteasomal degradation.

In order to find this cellular factor, non-permissive THP1 cells expressing Vpx fused to HA and Flag epitopes (eVpx) were generated.

The THP1 cells thus generated were lysed and thereafter a double immunopurification technique was then applied to the cell lysate using antibodies directed against the Flag epitope, followed by a second immunopurification with the use of antibodies specific to the HA epitope.

The products of immunoprecipitation were analysed on polyacrylamide gel followed by protein staining with Coomassie Blue dye. The proteins interacting with eVpx were identified by mass spectrometry.

The SAMHD1 protein was then identified as a major Vpx interactant.

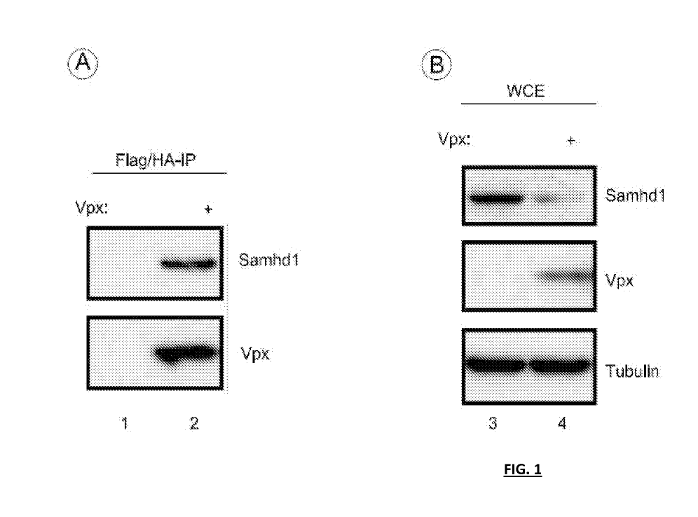

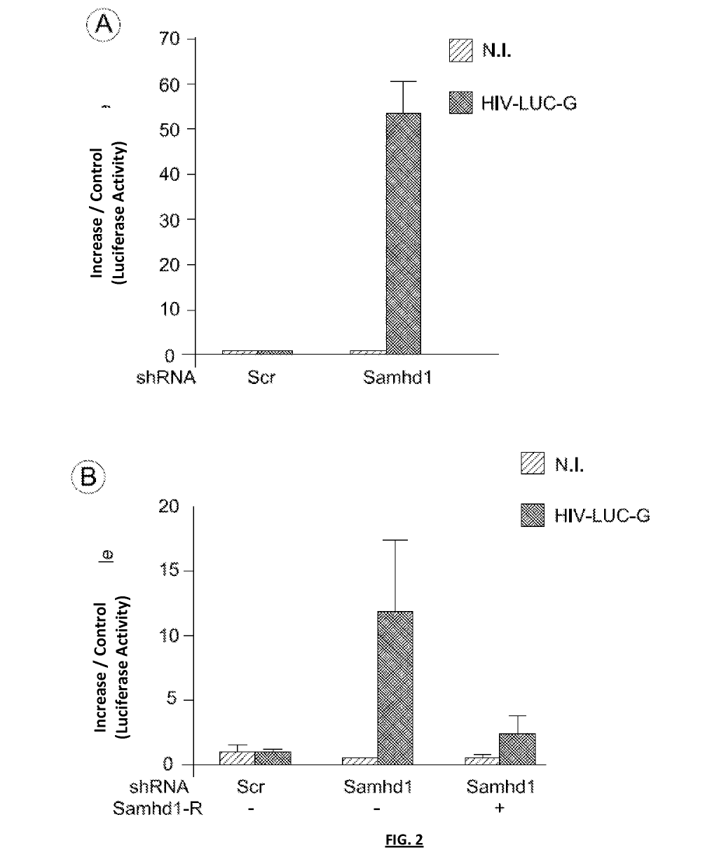

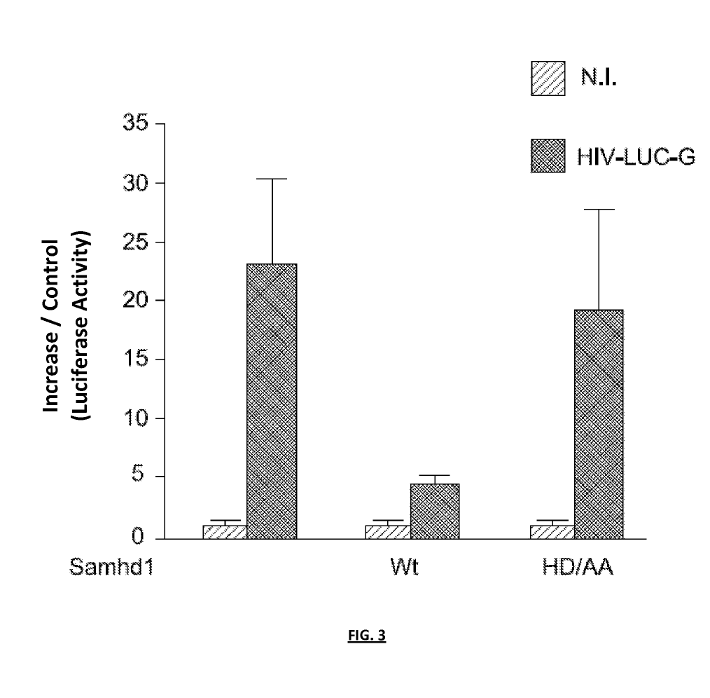

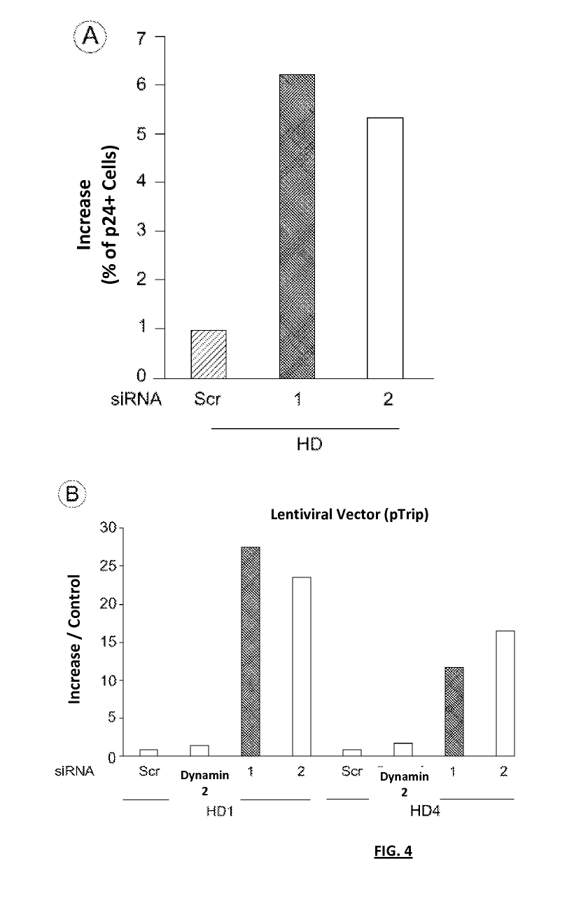

This interaction was validated by immunoprecipitation of the Vpx protein followed by detection of the SAMHD1 protein by using specific antibodies. For this purpose, non permissive THP1 cells were transduced with an expression vector expressing Flag and HA tagged Vpx (eVpx) (see FIG. 1, lane 2) or an empty vector (FIG. 1, lane 1).

The cell extracts were prepared and subjected to a double immunoprecipitation reaction with the use of Flag and HA tag specific antibody. The immunoprecipitates were analysed on acrylamide gel and the presence of SAMHD1 was tested by Western blot analysis by using an SAMHD1 specific antibody (see FIG. 1. A). The total cell extracts (“WCE”) prepared from cells expressing (see FIG. 1, lane 4) or not expressing (see FIG. 1, lane 3) eVpx were analysed on acrylamide gel followed by a Western blot analysis using anti-SAMHD1, Flag or tubulin antibody (see FIG. 1. B) as indicated.

The immunoprecipitation results are presented in FIG. 1.

FIG. 1 shows that Vpx interacts with the endogenous SAMHD1 protein (see FIG. 1. A). Quantification of SAMHD1 in extracts of cells expressing or not expressing Vpx also demonstrates that Vpx induces the degradation of SAMHD1 (FIG. 1. B).

Experiments were then conducted for the purposes of determining whether inhibition of the expression of SAMHD1 with the use of a specific shRNA renders the THP1 cells permissive to infection by the HIV-1 virus.

In order to do this, the THP1 cells were transduced with a control shRNA (Scr) (negative control) or an SAMHD1 specific shRNA so as to inhibit its expression. The transduced THP1 were infected with HIV-1 expressing luciferase (HIV-LUC-G column) or non infected (“NI” column). The infection was quantified by determination of luciferase activity in the cell extracts.

The results are presented in FIG. 2.

Inhibition of the expression of SAMHD1 by RNA interference (RNAi) in cells that are non-permissive to HIV-1 (cells of the differentiated human monocytic cell line THP1) renders them permissive thereto (FIG. 2. A). The overexpression of a form of SAMHD1 resistant to RNAi (SAMHD1-R protein) in cells where the level of SAMHD1 has been reduced by RNAi enables these cells to recover their phenotype of HIV non permissiveness (FIG. 2. B).

The effect of overexpression of SAMHD1 on the infection of cells that do not constitutively express this protein, namely HeLa cells, was then evaluated.

For this purpose, HeLa cells were transfected with an empty vector or a vector expressing the SAMHD1 protein or a SAMHD1 mutated within its “HDc” domain exhibiting phosphohydrolase activity (RNase) (Samhd1-HD/AA). The transfected cells were infected with the HIV-1 expressing luciferase. The infection of these cells was measured by assaying the luciferase activity.

The results are presented in FIG. 3.

This figure shows that overexpression of SAMHD1 in cells permissive to HIV-1 (HeLa cells) renders them non-permissive (FIG. 3, “WT” column). In an interesting manner, the overexpression of a mutated form of SAMHD1 not exhibiting phosphohydrolase activity has no effect on viral replication (FIG. 3, “HD/AA” columns). This result shows the importance of the enzymatic activity of SAMHD1 (phosphohydrolase activity) in the restriction of HIV-1.

The involvement of SAMHD1 in primary dendritic cells derived from mononuclear cells isolated from healthy donor blood was also tested.

For this purpose, peripheral cells from the blood of a healthy donor (HD) were purified and differentiated into dendritic cells ex vivo. These cells were treated with two different siRNAs specifically targetting SAMHD1 (siRNA corresponding respectively to SEQ ID NOs: 3 and 4) (see FIG. 4, columns 1 and 2, respectively), or were non treated (line Scr), then the percentage of p24 positive cells was determined by flow cytometry.

These results are presented in FIG. 4. A.

This figure shows that treatment with either of the siRNAs specifically targetting SAMHD1 results in an increase in the production of Gag (p24) within the cells following the infection with wild type virus, which is reflected in an increase in the viral transduction and infectivity of primary cells by HIV-1.

Similar experiments were conducted involving the use of primary dendritic cells derived from monocytic cells isolated from the blood of two healthy donors (“HD1” and “HD4”) by using a lentiviral vector pTRIP previously described by Royer-Leveau et al. (Journal of Virological Methods, 105: 133-140, 2002).

These results are presented in FIG. 4. B.

This figure clearly shows that the infection by the vector pTRIP is greatly increased in the primary dendritic cells of the two healthy donors, and that this is so regardless of which of the siRNAs specifically targetting SAMHD1 is used. This result indicates that the transduction of the lentiviral vector pTRIP is increased in dendritic cells when the expression of SAMHD1 is inhibited.

In conclusion, the SAMHD1 protein was identified as the restriction factor restricting infection by lentiviruses that is expressed by dendritic cells and macrophages and whose activity is counteracted by Vpx. Dendritic cells and macrophages play a crucial role in the development of the immune response. They are antigen presenting cells. They are necessary in order to ensure a coordinated and sustainable response thereby enabling protection of the body against infectious agents. Several studies suggest that the ineffectiveness of current vaccines is mainly due to the restriction of HIV-1 infection of dendritic cells and macrophages. The identification of SAMHD1 as the restriction factor expressed by these cells is therefore crucial for the development of an effective vaccine against lentiviruses in general and the HIV-1 virus in particular. Furthermore, this discovery allows for improving the transduction of cells expressing the SAMHD1 protein by lentiviral vectors for the purposes of gene therapy or for in vitro experiments.

1. An in vitro method of screening candidate compounds capable of being used for the preventative and/or curative treatment of a disease caused by a retrovirus, comprising the following steps:
contacting a candidate compound with a cell expressing SAMHD1 or with the SAMHD1 protein; and
determining whether the candidate compound modulates the expression and/or the activity of the SAMHD1 protein;
wherein said candidate compound which modulates the expression and/or the activity of the SAMHD1 protein is identified as a candidate compound capable of being used for the preventive and/or curative treatment of a disease caused by a retrovirus.

2. An in vitro method of screening according to claim 1, characterised in that:
the determination of the ability of the candidate compound to modulate the expression of SAMHD1 is carried out by quantifying the SAMHD1 mRNA and/or the SAMHD1 protein; and
the determination of the ability of the candidate compound to modulate the activity of SAMHD1 is carried out by measuring the phosphohydrolase activity of SAMHD1.

3. An in vitro method of screening according to claim 1, characterised in that the retrovirus is a retrovirus belonging to a genus selected from the group consisting of the Alpharetroviruses, the Betaretroviruses, the Gammaretroviruses, the Deltaretroviruses, the Epsilonretroviruses, the Lentiviruses and the Spumaviruses.

4. An in vitro method of screening according to claim 1, characterised in that the retrovirus belongs to the genus Lentivirus and is selected from the group constituted of species consisting of human immunodeficiency viruses type 1 (HIV-1), human immunodeficiency viruses type 2 (HIV-2) and simian immunodeficiency viruses (SIV).

5. A method of treating and/or preventing a disease caused by a retrovirus in a subject in need thereof, wherein the method comprises a step consisting of administering to the subject a modulator of the expression and/or the activity of SAMHD1.

6. The method according to claim 5, characterised in that the retrovirus which causes the disease is a lentivirus, in particular the species consisting of human immunodeficiency viruses type 1 (HIV-1), human immunodeficiency viruses type 2 (HIV-2) and simian immunodeficiency viruses (SIV).

7. The method according to claim 5, characterised in that the modulator is selected from the group consisting of a small chemical molecule, an interfering RNA directed against the SAMHD1 mRNA or pre-mRNA, an antisense oligonucleotide targetting the gene encoding SAMHD1, a ribozyme directed against the SAMHD1 mRNA or pre-mRNA, an anti-SAMHD1 antibody, or an anti-SAMHD1 antibody derivative, and an aptamer.

8. An immunogenic or vaccine composition comprising:
i) an inhibitor of the expression and/or activity of the SAMHD1 protein; and
ii) at least one antigen of a retrovirus or a polynucleotide sequence encoding this antigen.

9. An immunogenic or vaccine composition according to claim 8, characterised in that the antigen is selected from the group consisting of: a protein of said retrovirus or a fragment thereof, an expression vector comprising the polynucleotide sequence encoding a protein of said retrovirus or a fragment thereof, a virus like particle having the structural proteins of said retrovirus, a lipopeptide antigen comprising proteins or peptide fragments of said retrovirus, said retrovirus in its inactivated form or in its attenuated form, and a pseudotyped viral vector.

10. An immunogenic or vaccine composition according to claim 8, characterised in that the inhibitor is selected from the group consisting of anti-SAMHD1 antibodies, interfering RNAs directed against the SAMHD1 mRNA or pre-mRNA, antisense oligonucleotides targetting the gene encoding SAMHD1, ribozymes directed against the SAMHD1 mRNA or pre-mRNA and aptamers.

11. An immunogenic or vaccine composition according to claim 8, characterised in that the retrovirus is a human immunodeficiency virus type 1 (HIV-1).

12. Method of in vitro or ex vivo increasing the transduction of cells expressing SAMHD1 by a retroviral vector, the method comprises a step wherein the cells are contacted with an inhibitor of the expression and/or the activity of the SAMHD1 protein.

13. The method according to claim 12, characterised in that the retroviral vector is a human immunodeficiency virus type 1 (HIV-1), a human immunodeficiency virus type 2 (HIV-2), or a simian immunodeficiency virus (SIV).

14. A kit for transducing a retroviral vector in a cell naturally expressing SAMHD1, comprising:
an inhibitor of the expression and/or the activity of the SAMHD1 protein;
a retroviral expression vector that does not naturally express the Vpx protein.

15. In vitro or ex vivo method of inducing the expression of type 1 interferons by a dendritic cell and/or a cell of the monocytic lineage, the method comprises a step wherein the dendritic cell and/or the cell of the monocytic lineage are contacted with an inhibitor of the expression and/or the activity of the SAMHD1 protein.

16. A method of inducing expression of type 1 interferons by a dendritic cell and/or a cell of the monocytic lineage in a subject in need thereof, the method comprises a step wherein the subject is administered with an inhibitor of the expression and/or the activity of the SAMHD1 protein.

17. An in vitro or ex vivo method for inducing resistance to infection by a retrovirus, in a cell that does not naturally express the SAMHD1 protein, said method comprising a step consisting of introducing a sequence encoding the SAMHD1 protein in the said cell that does not naturally express the SAMHD1 protein, the cells expressing the SAMHD1 protein being resistant to infection by a retrovirus.

18. A method of treating or preventing a disease caused by a retrovirus in a subject in need thereof, the methode comprises a step wherein a cell not naturally expressing the SAMHD1 protein in which the sequence encoding the SAMHD1 protein has been introduced is administered to the subject, said cell being a target cell of the retrovirus during infection or a cell that is capable of differentiating into a target cell of the retrovirus.

19. The method according to claim 18, characterised in that the cell not naturally expressing the SAMHD1 protein in which the sequence encoding the SAMHD1 protein has been introduced is derived from the subject.

20. An in vitro or ex vivo method according to claim 17, wherein the retrovirus is an HIV-1 virus.

21. An in vitro or ex vivo method according to claim 17, wherein the cell not naturally expressing the SAMHD1 protein is a CD34+ stem cell capable of differentiating into CD4+ T lymphocytes.

22. An in vitro or ex vivo method according to claim 17, wherein the introduction of the sequence of the gene encoding SAMHD1 in said cell not naturally expressing the SAMHD1 protein is carried out by using a gene transfer vector.

23. A method according to claim 18, wherein the retrovirus is an HIV-1 virus.

24. A method according to claim 18, wherein the cell not naturally expressing the SAMHD1 protein is a CD34+ stem cell capable of differentiating into CD4+ T lymphocytes.

25. A method according to claim 18, wherein the introduction of the sequence of the gene encoding SAMHD1 in said cell not naturally expressing the SAMHD1 protein is carried out by using a gene transfer vector.

26. A cell that does not naturally express the SAMHD1 protein, characterised in that it expresses a recombinant SAMHD1 protein.


SEQUENCE LISTING (6 sequences)

SEQ ID NO: 1 - protein, 626 aa, Homo sapiens
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVINMDYGMQEKNPIDHVSFYCKTAPNRAIRITKNQVSQLLPEKFAEQLIRVYCKKVDRKSLYAARQYFVQWCADRNFTKPQDGDVIAPLITPQKKEWNDSTSVQNPTRLREASKSRVQLFKDDPM

SEQ ID NO: 2 - DNA, 3189 bp, Homo sapiens
  misc_feature (201)..(2081): CDS
attgcgcctgcgcagggagcccaaggcaagagccgctaggctgccctgcccgaagggctcaactgtcagtgagcctgcgcaggaggccaataggctgccaatactccttggactccccgccagggccctgctgtcagtgcgcctgcgcgcgggtccggcgccgaggttcttgactgctgtgccggacgccaggtgtagccatgcagcgagccgattccgagcagccctccaagcgtccccgttgcgatgacagcccgagaaccccctcaaacaccccttccgcagaggcagactggtccccgggcctggaactccatcccgactacaagacatggggtccggagcaggtgtgctccttcctcaggcgcggtggctttgaagagccggtgctgctgaagaacatccgagaaaatgaaatcacaggcgcattactgccttgtcttgatgagtctcgttttgaaaatcttggagtaagttccttgggggagaggaagaagctgcttagttatatccagcgattggttcaaatccacgttgatacaatgaaggtaattaatgatcctatccatggccacattgagctccaccctctcctcgtccgaatcattgatacacctcaatttcaacgtcttcgatacatcaaacagctgggaggtggttactatgtttttccaggagcttcacacaatcgatttgagcatagtctaggggtggggtatctagcaggatgtctagttcacgcactgggtgaaaaacaaccagagctgcagataagtgaacgagatgttctctgtgttcagattgctggactttgtcatgatctcggtcatgggccattttctcacatgtttgatggacgatttattccacttgctcgcccggaggtgaaatggacgcatgaacaaggctcagttatgatgtttgagcaccttattaattctaatggaattaagcctgtcatggaacaatatggtctcatccctgaagaagatatttgctttataaaggaacaaattgtaggaccacttgaatcacctgtcgaagattcattgtggccatataaagggcgtcctgaaaacaaaagcttcctttatgagatagtatctaataaaagaaatggcattgatgtggacaaatgggattattttgccagggactgccatcatcttggaatccaaaataattttgattacaagcgctttattaagtttgcccgtgtctgtgaagtagacaatgagttgcgtatttgtgctagagataaggaagttggaaatctgtatgacatgttccacactcgcaactctttacaccgtagagcttatcaacacaaagttggcaacattattgatacaatgattacagatgctttcctcaaagcagatgactacatagagattacaggtgctggaggaaaaaagtatcgcatttctacagcaattgacgacatggaagcctatactaagctgacagataacatttttctggagattttatactctactgatcccaaattgaaagacgcacgagagattttaaaacaaattgaataccgtaatctattcaagtatgtgggtgagacgcagccaacaggacaaataaagattaaaagggaggactatgaatctcttccaaaagaggttgccagtgctaaacccaaagtattgctagacgtgaaactgaaggctgaagattttatagtggatgttatcaacatggattatggaatgcaagaaaagaatccaattgatcatgttagcttctattgtaagactgcccccaacagagcaatcaggattactaaaaaccaggtttcacaacttctgccagagaaatttgcagagcagctgattcgagtatattgtaagaaggtggacagaaagagtttgtatgccgcaagacaatattttgttcagtggtgtgcagacagaaatttcaccaagccgcaggatggcgatgttatagccccactcataacacctcaaaaaaaggaatggaacgacagtacttcagtccaaaatccaactcgcctccgagaagcatccaaaagcagagtccagctttttaaagatgacccaatgtgaatgtctgtagtcagttgtttacaaactccctctcctgcacaattcatttagaggcttcaatcatagaattctgcaaattaatgacaactcatgctttaattttgtattttgaatgtacacgcatgctgaagctaagtaacttttaatcaaagaaataagatggtattaggcaaatcttactatactatgaaaagcattaccttgcctatttttaatattattaaagcctttctccttcagtagtctattttcttagaataacaactcttttatctattctgaactctattttttttcttttttaagagacaaggttttgctctgttgcccagcttggactcgaactttcctgggctcaagcgaccctcctgcctcagccccccaagtagctgggactaaagtcatgtgccaccacacccagcttactctgaacttttatgacagatgattgttttttgtttttaatgtagaaatgagacaagggtacaaattggaactagaaattgacattgtcattgacaaacatggctaaaaacaaaacatcaaatcctgcccccgtgaagagttccctgtcacctcaagtttgaggatagtcctctaagagtgacctaagcataagtgaaagacacctcccctcacccttctagccccctacaaggtgccaggttggggtaaagagttggagatgatggccaggagtggcctccaacacgctggtgagaggcctgattaggttttggggaagatctgagagctctggcctcttcgtgagtggaacataaagccgcctcttgttgggagatcctaccccagtgacagaggaatcccccaaactaggctgtgccctggctccgtggcggctccagacccgggtagtgcctttgtcccctgaatactcactcccccggtccagagggccttcccactgcccagcctggagaaggcctcccctgacctgctctctcagtatcctggagagctggccagaggccatcacaggcatcatcctcagagctcctcagacctgggactttgtttttgctggttcagtgcattttgtgtatttaagagcaaacactagccaggcgtggcggcgtgtgcctgtagt

SEQ ID NO: 3 - RNA, 19 nt, Artificial Sequence
  samhd1 siRNA
gauucauuguggccauaua

SEQ ID NO: 4 - RNA, 19 nt, Artificial Sequence
  samhd1 siRNA
caaccagagcugcagauaa

SEQ ID NO: 5 - RNA, 19 nt, Artificial Sequence
  samhd1 shRNA (sens)
cgcaggauggcgauguuau

SEQ ID NO: 6 - RNA, 19 nt, Artificial Sequence
  samhd1 shRNA (antisens)
auaacaucgccauccugcg